(19) United States
(12) Patent Application Publication
(10) Pub. No.: US 2015/0331073 A1
(43) Pub. Date: Nov. 19, 2015

(54) MAGNETIC RESONANCE TOMOGRAPHY APPARATUS AND METHOD FOR ASSISTING A PERSON WHEN POSITIONING A MEDICAL INSTRUMENT FOR A PERCUTANEOUS INTERVENTION

(71) Applicant: Siemens Aktiengesellschaft, Muenchen (DE)

(72) Inventors: Wesley David Gilson, Pasadena, MD (US); Eva Rothgang, Nuernberg (DE)

(73) Assignee: Siemens Aktiengesellschaft, Muenchen (DE)

(21) Appl. No.: 14/713,375

(22) Filed: May 15, 2015

(30) Foreign Application Priority Data
May 16, 2014 (DE) 102014209368.6

(51) Int. Cl. G01R 33/28; G01R 33/56; A61B 19/00; A61B 5/00; A61B 5/055; A61B 10/02

(52) U.S. Cl. CPC G01R 33/286; A61B 5/055; A61B 10/0233; A61B 19/54; A61B 5/7425; G01R 33/5608; A61B 2019/5454

(57) ABSTRACT

A medical instrument for carrying out a percutaneous intervention in a patient is provided with a marker that is visible in an MR image. A real-time magnetic resonance image of the patient is created, so that the actual position of the marker can be identified in the real-time image. For assisting a person in the positioning of the medical instrument in an initial position suitable for the intervention, a desired position of the marker that correlates with the initial position is displayed in the real-time image. The positioning thus can be carried out relatively effortlessly and quickly.

### BACKGROUND OF THE INVENTION

1. Field of the Invention

The invention concerns a magnetic resonance tomography apparatus (MRT apparatus) for assisting a person when positioning a medical instrument for implementing a (MR-guided) percutaneous intervention in a patient. The invention also concerns a method for supporting such a person when positioning of such a medical instrument for implementing such a percutaneous intervention.

2. Description of the Prior Art

A percutaneous intervention is a medical intervention in which a medical instrument is introduced into the body of a patient so as to be as minimally invasive as possible. The aim of the intervention is usually to reach a lesion (abnormal tissue) inside the body with the medical instrument. The medical instrument is typically a needle or cannula or the like. Examples of such percutaneous interventions are biopsies, thermal ablations or local applications of drugs.

To enable precise guidance of the instrument to the lesion in the body, imaging methods for supporting the person carrying out the intervention (hereinafter also called: “operator” for short) are conventionally used. A real-time image of the inside of the body or a body segment of the patient is conventionally displayed for the operator, so that the operator can follow the path of the medical instrument inside the body. Magnetic resonance tomography (MRT) is increasingly being used for real-time imaging, since lesions can be identified particularly well by the outstanding soft tissue contrast of MRT. Sometimes lesions can even be identified solely by means of MRT.

MR imaging has the drawback, however, that the medical instrument used for the intervention cannot be seen outside of the body in the real-time image. The operator must therefore bring the medical instrument more or less “blind” into a position in which the instrument is suitably directed toward the lesion.

Correct orientation of the medical instrument usually occurs therefore only after the entry thereof into the body and this can lead to unnecessary damage to body tissue. Furthermore, there are different tracking methods for the instrument, although these are associated with comparatively high outlay in teems of apparatus components.

### SUMMARY OF THE INVENTION

An object of the invention is to assist a person when positioning a medical instrument for implementing an MR-guided (i.e. guided by magnetic resonance tomography) percutaneous intervention, so as to indicate a pre-operative initial position suitable for the intervention. It should be possible to carry out the positioning relatively easily and quickly.

A magnetic resonance tomography apparatus for assisting a person (an operator) when positioning a medical instrument for implementing a percutaneous intervention in a patient includes a marker that is visible in a magnetic resonance image and that is provided on the medical instrument.

Furthermore, the MRT system has a magnetic resonance tomography scanner (MRT scanner). The MRT scanner is operated to produce a real-time image of the patient, so that—at least if the medical instrument is located in a region intended for the intervention—the marker can be seen in the real-time image.

For assisting the operator in the pre-operative orientation of the medical instrument (generally not visible outside of the patient's body in the real-time image), the MRT system also has a processor configured to display in the real-time image a desired position of the marker that correlates with a predefined initial position of the medical instrument.

For correct positioning of the instrument, the operator must then orient the medical instrument in space so that the actual position of the marker is matched to the desired position that is likewise displayed in the real-time image.

An inventive method for assisting a person when positioning a medical instrument for implementing an MR-guided percutaneous intervention in a patient, included the steps of providing the medical instrument with a marker that is visible in an MR image. In principle this can be done in advance by an instrument manufacturer, but this step is preferably done by the operator during the course of preparation for the intervention. A real-time image of the patient is then generated by MRT, in which the actual position of the marker can be seen with at least approximately correct positioning of the medical instrument. The desired position of the marker that correlates with a predefined initial position of the medical instrument is also displayed in the real-time image, so that the operator can bring the medical instrument into the desired initial position by comparison of the actual position of the marker with the desired position of the marker.

The method can advantageously be applied to any MR-compatible instrument. The method can be applied particularly advantageously to manually-guided instruments, since then the low additional outlay for implementing the method is shown to particular advantage. All that is necessary is for the instrument to be provided with the marker and a software program for displaying the desired position in the real-time image to be installed on a processor so as to be executable.

At least one section of the medical instrument can already be seen outside of the body in the MR image due to the marker. The operator can consequently advantageously already precisely position the instrument before it enters the body. The orientation of the instrument can be carried out easily and intuitively hereby.

Within the context of the invention “marker” generally designates an object which is made at least partly from a material whose nuclear spins can be excited by the MRT device, and which can therefore be seen in an MR image. In a preferred embodiment the instrument is a thin, elongated instrument that is preferably (but not exclusively) suitable for carrying out a biopsy, for carrying out a thermal ablation or for carrying out a local drug application. In particular the instrument can be a needle, electrode or cannula appropriate to the respective application.

An image or MR image designates a depiction which is produced from measurement data acquired by the MRT device. The MR image (the depiction) is expediently displayed on a display unit that is situated in the vicinity of the MRT device.

An MR image is characterized as a “real-time” image when the image is produced at a scan rate that is sufficiently high for online tracking of the actual position of the marker. The image produced is updated at a scan rate of, for example, two images or more per second.

Within the context of the invention the term “initial position” designates a position of the medical instrument in space that is an insertion position or entry position for the medical instrument according to an intervention plan produced before the procedure. This means the initial position describes the position of the instrument immediately before the start of the intervention. The initial position is chosen such that a longitudinal extension of the instrument aligns with a planned intervention path. The initial position is preferably fixed by specifying two points of the instrument. The first point can be the instrument tip, which in the initial position is placed at a predefined entry point in the body. To fix the second point the marker is provided on the instrument at a defined spacing from the instrument tip. The marker is small compared with the longitudinal extent of the instrument. It is also within the context of the invention, however, for multiple markers to be provided on the instrument, so then for orientation of the instrument all of the markers have to be brought into an appropriate desired position displayed in the real-time image.

Within the context of the invention, the desired position to be schematically can be shown in the real-time image solely by a marking point. In a preferred embodiment, however, an external contour of the marker is displayed at the desired position by the processor, so that the overlaying of the actual position of the marker with its desired position may advantageously be carried out particularly precisely.

For additional assistance, the processor can be configured to incorporate the geometry of the instrument provided with the marker, to determine the location of the desired position from a predefined entry point of the instrument in the body of the patient and a predefined target point inside the body of the patient. Entry point and target point are fixed by the operator during the course of intervention planning using a previously acquired image of the patient. For example, the operator can “click” the entry point and the target point in a planning image, from which the processor first determines the respective positions in space and from this determines the position of the marker in space by incorporating the geometry of the instrument provided with the marker. The initial position is specified such that a longitudinal orientation of the instrument—if it is located with its tip at the entry point—aligns precisely with an intervention path that leads from the entry point to the target point.

For particularly straightforward display and overlaying (matching), the marker is rotationally symmetrical in design, with its axis of symmetry preferably oriented coaxially to a longitudinal extent of the instrument. The marker is preferably approximately spherical in design. The marker is preferably small in dimension compared to the longitudinal extent of the medical instrument.

The marker preferably is a hollow body that contains a medium that can be depicted by the MRT device, in particular water to which gadolinium has been added, or Vitamin E.

For provision of the marker on the medical instrument, the marker preferably has a passage, such as a central passage, with which it can be placed on the medical instrument. The passage is dimensioned such that the marker can be held in place on a port of a cannula or directly on a cannula of the medical instrument.

The marker is preferably in the form of a component part that is separate from the instrument, with the marker only being provided on the instrument only by the operator. Commercially available medical instruments can advantageously be used hereby for the MRT system described above.

### BRIEF DESCRIPTION OF THE DRAWINGS

FIG. 1 schematically illustrates an MRT apparatus for assisting a person when positioning a medical instrument for a percutaneous intervention in a patient.

FIG. 2 schematically illustrates the medical instrument provided with a marker that can be depicted in an MR image.

FIG. 3 also shows the marker according to FIG. 2.

FIG. 4 shows a real-time image produced with the MRT apparatus of FIG. 1 according to the invention.

FIG. 5 is a flowchart of a method for assisting a person when positioning a medical instrument for a percutaneous intervention in a patient according to the invention.

### DESCRIPTION OF THE PREFERRED EMBODIMENTS

Corresponding parts are provided with the same reference characters in all figures.

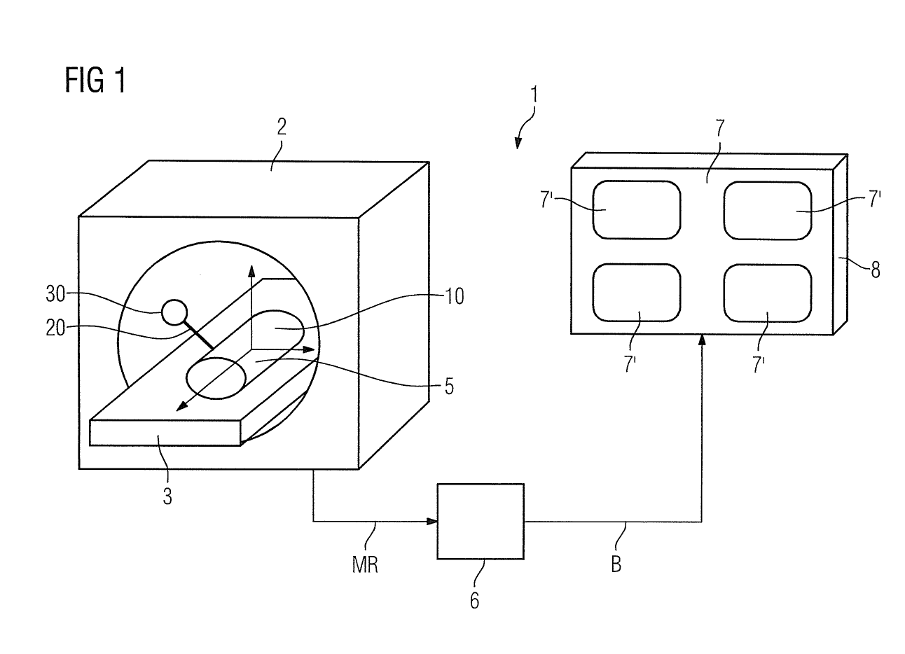

FIG. 1 shows schematically (and not-to-scale) illustrates a magnetic resonance tomography apparatus (MRT apparatus 1 for short), having a magnetic resonance tomography scanner 2 (MRT scanner 2 for short). A patient bed 3 for supporting a person to be examined or treated (hereinafter “patient 5”) is associated with the MRT scanner 2. The MRT system 1 also has a processor 6, which is used for operating the MRT scanner 2 and for causing a magnetic resonance image (MR image 7), reconstructed in the processor 6, to be shown on a display unit 8.

The MRT scanner 2 is constructed in a conventional manner. It has a basic field main magnet for generating a basic magnetic field, radio-frequency coils for resonant excitation of nuclear spins of certain body tissue of the patient 5, and a gradient coil system for spatial resolution of the magnetic resonance signal (MR signal MR) resulting from the resonant excitation. A coordinate system 10 of the scanner 2 is defined by the gradient coil system. Three coordinates of the coordinate system 10 are clearly associated with each volume element of the acquired MR data.

For image generation, the processor 6 derives an image data record B from the MR signal. An image point (voxel) of the image data record B is associated with each volume element considered (defined by its 3D coordinates). From the MR signal MR the processor 6 determines for each image point a gray scale value that represents the tissue properties of the associated volume element.

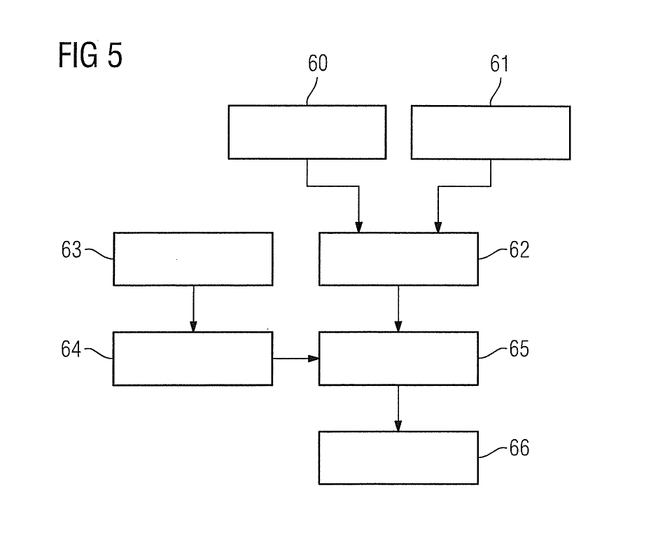

The processor 6 produces one or more two-dimensional MR image(s) 7 (e.g. in the form of sectional views or rendered scenes) from the three-dimensional image data record B and emits electronic signals represented by each MR image 7 to the display unit 8 of the MRT systems 1 as the MR image 7. Different views in particular 7′ can be produced from a single image data record B. FIG. 4 shows a sectional view of this as an example.

In the present case the MRT system 1 is used for supporting a percutaneous intervention in the patient 5. The example of a biopsy as the intervention is used below. A tissue sample in the region of a lesion inside the body of the patient 5 is to be extracted using a medical instrument 20. The person carrying out the biopsy will be called the “operator” below.

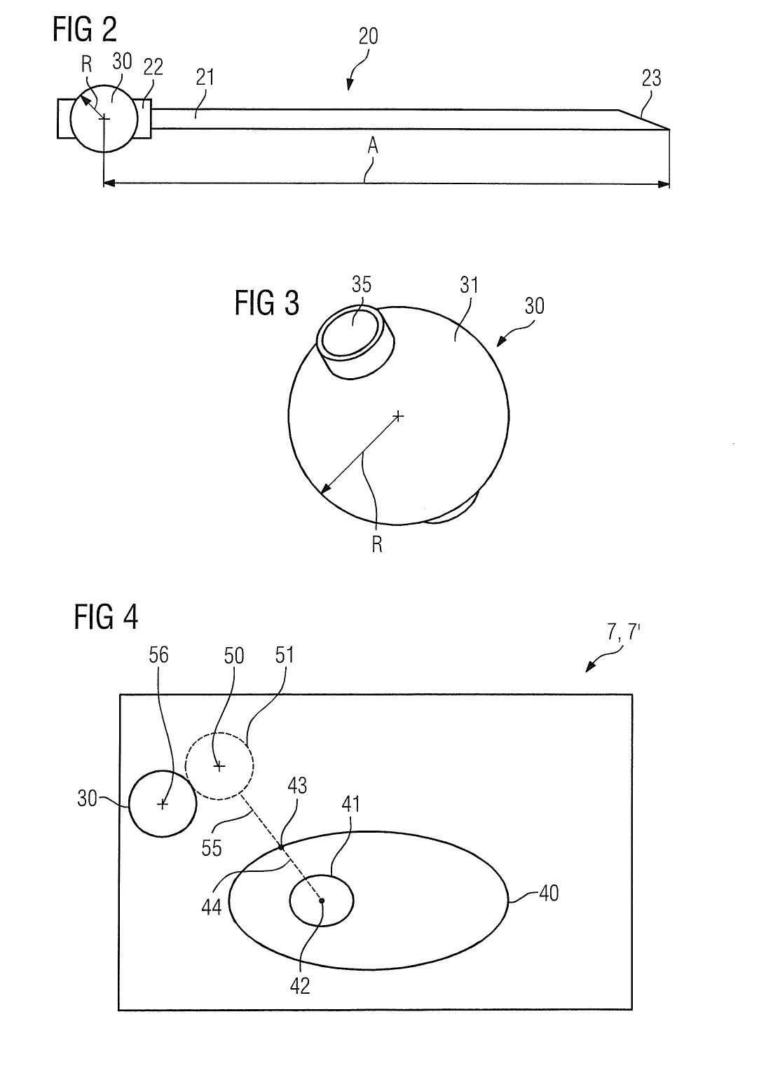

The medical instrument 20 is shown in FIG. 2 in a side view. The instrument is a commercially available, MR-compatible (biopsy) needle. The instrument 20 is formed by a cannula 21 with a connecting element 22 made from plastic. The connecting element 22 is used to conventionally connect the cannula 21 to a vacuum device for generating suction for removal of tissue. A tip 23 is formed on the cannula 21 at the longitudinal end that faces the connecting element 22. Connecting to the vacuum device is optional, however. The instrument 20 can alternatively also be designed as a biopsy needle, which cuts or punches out the tissue sample to be removed without the application of a vacuum.

The instrument 20 itself cannot be depicted by MRT and is therefore not visible in the MR image 7 outside of the body. The instrument 20 can only be seen inside the body for the MRT as a consequence of susceptibility artifacts produced thereby. A marker 30 that can be depicted by MRT is nevertheless provided on the connecting element 22.

The marker 30 (shown in a perspective view in FIG. 3) has an approximately spherical hollow body 31 having a defined spherical radius R of, for example, approximately 0.5 cm. The hollow body 31 is filled with a medium that can be depicted by MR, in this case with vitamin E. The wall of the hollow body 31 is made from a rubbery material. The marker 30 is placed as intended on the connecting element 22 of the instrument 20 (see FIG. 2) with a continuous central passage 35. The diameter of the passage 35 is dimensioned such that the marker 30 is held by friction on the connecting element 22. The center of the marker is then at a defined spacing A from the tip 23. In an alternative embodiment the diameter is dimensioned such that the marker 30 can be placed on the cannula 31.

FIG. 4 shows the image data record B in one of the views 7′ according to FIG. 1, with a section through the body 40 of the patient 5 being shown here.

A (suspected) lesion 41 can be seen inside the body 40. Shown in the region of the lesion 41 is a target point 42 at which the tissue sample is to be removed. Fixed on the surface of the body is an entry point 43 at which the instrument 20 should be introduced into the body 40. The depiction of entry point 43, target point 42 and intervention path 44 in the MR image 7 is optional.

In any case, a contour 51 corresponding to the marker dimensioning is overlaid on the MR image 7 at a desired position 50. The desired position 50 for supporting the operator in the positioning of the medical instrument 20 represents the position that the marker 30 adopts if the instrument 20 is located in an initial position 55 (likewise optionally depicted in the MR image 7) with its tip 23 at the entry point 43 and oriented in the direction of the intervention path 44.

As can be seen from FIG. 4, a depiction of the marker 30 can be seen in the MR image 7 moreover, and, more precisely, at its actual position 56 at which it is currently located in the position of the instrument 20 shown according to FIG. 1.

With knowledge of the desired position 50, the operator is able to orient the instrument 20 in the desired initial position 55 by moving the instrument 20 in space, with simultaneous MR imaging, until the current depiction of the marker 30 covers the contour 51 (the “virtual image” of the marker 30) at its desired position 50.

A method for assisting a person in the positioning of the medical instrument 20 is explained using the flowchart in FIG. 5.

In a first step 60 the operator carries out an intervention plan in preparation for the biopsy. Using either the MRT scanner 2 or another modality, an image of the patient 5 is recorded in advance, in which the lesion 41 to be treated can be seen. The operator fixes target point 42 (FIG. 4) and entry point 43 (FIG. 4) by “clicking” or some other form of marking in this image. Using the marked image points the processor 6 determines the 3D coordinates of target point 42 and entry point 43 within the coordinate system 10.

In a second step 61 the operator chooses a suitable medical instrument 20 for carrying out the intervention (by way of example the needle according to FIG. 2) and provides this with the suitable marker 30. The spacing A and the spherical radius R are fed to the processor 6 as geometric data of the medical instrument 20 provided with the marker 30. The operator inputs the data manually by way of example, or he has the option of choosing the instrument 20 and the marker 30 from a list, with the associated geometric data A, R being retrievably stored for the processor 6.

In a further step 62, the processor 6 determines from the 3D coordinates of target point 42 and entry point 43, as well as the spacing A firstly the 3D coordinates of the desired position 50 at which the center of the sphere of the marker 30 must be located if the instrument 20 is oriented in the initial position 55. Furthermore, the processor 6 determines the position of the contour 51 with spacing R from the desired position 50.

During the course of the actual intervention the operator is then firstly assisted in step 63, as is known, for example, from US 2013/0218003 A1, in finding the entry point 43 on the body of the patient 5. Alternatively the operator finds the entry point 43 in an image-assisted manner by placing a finger (which can be depicted by MR) or by MR-visible marking points which are provided on the skin of the patient 5 in the region of the anticipated entry point 43. Once the entry point 43 has been found and prepared for the intervention the operator places the instrument 20 with its tip 23 at the entry point 43 (according to the diagram in FIG. 1).

In step 64, the processor 6 activates the MRT device 2 to start a data acquisition protocol that is capable of producing the MR image 7 in real-time. A data acquisition protocol of this kind is, for example, a balanced SSFP sequence (“Steady State Free Precession”).

Finally, in step 65 the processor 6 combines the MR signal MR of the MRT device 2 with the determined desired position 50. The processor 6 generates an image data record B in which the contour 51 with spacing R from the desired position 50 is provided. In other words, the processor 6 synthetizes a virtual image of the marker 30, specifically of its external contour, and overlays this virtual image on the MR image at the calculated desired position 50.

In step 66, the MR image 7 modified in this way is shown to the operator on the display unit 8, so that the operator can then position the instrument 20 in the desired initial position 55 by optical feedback.

The operator first moves the instrument 20 until the depiction of the marker 30 appears in the MR image 7. The operator then performs the orientation within the cutting plane. Typically the operator has even more views 7′ of the image data record B available, however, wherein the actual position 56 of the marker 30 must then be aligned in all views 7′ with the desired position 50. The sectional images are ideally chosen such that the intervention path 44 is located in the image plane (analogously to FIG. 4).

In an alternative embodiment, the MR image 7 can be formed by two projections that are perpendicular to each other. As a further alternative, the MR image 7 can be a volume depiction.

Once the instrument 20 has been positioned, the tip 23 is finally guided—again with real-time imaging—along the intervention path 44 to the target point 42 and the tissue sample removed.

Although modifications and changes may be suggested by those skilled in the art, it is the intention of the inventors to embody within the patent warranted hereon all changes and modifications as reasonably and properly come within the scope of their contribution to the art.

We claim as our invention:

1. A magnetic resonance tomography apparatus comprising:
a magnetic resonance tomography scanner adapted to receive a patient;
a medical instrument designed to implement a percutaneous intervention in said patient, said instrument having a marker attached thereto comprising material that is visible in a magnetic resonance image;
a control computer configured to operate the MR tomography scanner during said percutaneous intervention to generate a real-time image of the patient, in which said marker is visible at an actual position of said marker in said real-time image;
a display monitor in communication with said control computer; and
said control computer being provided with a designation of a predetermined initial position of said instrument prior to insertion of the instrument into the patient in said percutaneous intervention, and said control computer being configured to cause said real-time image to be displayed at said display monitor with a position of said marker being designated in the real-time image that is correlated with said predetermined initial position of said instrument, and with said actual position of said marker also being shown in said real-time image at said display monitor.

2. A magnetic resonance tomography apparatus as claimed in claim 1 wherein said control computer is configured to display an external contour of the marker at said position at said display monitor correlated with said predetermined initial position of said instrument.

3. A magnetic resonance tomography apparatus as claimed in claim 1 wherein said control computer is configured to determine said position of said maker correlated with said predetermined initial position of said instrument by incorporating geometry of said instrument with said marker from a predetermined entry point of the instrument into the body of the patient, and a predetermined target point for said percutaneous intervention inside the body of the patient.

4. A magnetic resonance tomography apparatus as claimed in claim 1 wherein said medical instrument is selected from the group consisting of needles and cannulae.

5. A magnetic resonance tomography apparatus as claimed in claim 1 wherein said marker is rotationally symmetrical.

6. A magnetic resonance tomography apparatus as claimed in claim 5 wherein said marker is spherical.

7. A magnetic resonance tomography apparatus as claimed in claim 1 wherein said marker comprises a hollow body filled with a medium that is detectable in said magnetic resonance image.

8. A magnetic resonance tomography apparatus as claimed in claim 1 wherein said marker comprises a passage therein through which said instrument proceeds, to place and hold said marker on said instrument.

9. A method for assisting positioning of an instrument for implementing a percutaneous intervention in a patient, said method comprising:
placing a patient in a magnetic resonance tomography scanner;
providing a medical instrument, designed to implement a percutaneous intervention in said patient, with a marker attached thereto comprising material that is visible in a magnetic resonance image;
from a control computer, operating the MR tomography scanner during said percutaneous intervention to generate a real-time image of the patient, in which said marker is visible at an actual position of said marker in said real-time image, said control computer being in communication with a display monitor; and
providing said control computer with a designation of a predetermined initial position of said instrument prior to insertion of the instrument into the patient in said percutaneous intervention and, from said control computer, causing said real-time image to be displayed at said display monitor with a position of said marker being designated in the real-time image that is correlated with said predetermined initial position of said instrument, and with said actual position of said marker also being shown in said real-time image at said display monitor.

10. A method as claimed in claim 9 comprising displaying an external contour of said marker at said position on said display monitor correlated with said predetermined initial position of said instrument.

11. A method as claimed in claim 9 comprising determining said position of said marker on said display monitor correlated with said predetermined initial position of said instrument by incorporating geometry of said instrument with said marker from a predetermined entry point into the body of the patient and a predetermined target point of said percutaneous intervention inside the body of the patient.
